(19) United States
(12) Patent Application Publication
(10) Pub. No.: US 2004/0197801 A1
(43) Pub. Date: Oct. 7, 2004

(54) Nucleic acid sequence detection

(76) Inventors: Timothy Z. Liu, Fremont, CA (US); Scott Beavers, San Carlos, CA (US); Wanli Bi, San Ramon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/713,479

(22) Filed: Nov. 14, 2003

Related U.S. Application Data
(60) Provisional application No. 60/427,085, filed on Nov. 15, 2002.

(51) Int. Cl. C12Q 1/68; C12M 1/34

(52) U.S. Cl. 435/6; 435/287.2

(57) ABSTRACT

The present application discloses a system and a method for detecting a target nucleic acid sequence using a support comprising an electrode with a nucleic acid probe attached thereto. A nucleic acid target and a non-covalent photoelectrochemical label are contacted with the probe. The photoelectrochemical intercalator preferentially binds double-stranded nucleic acids. Irradiating the mixture generates a photocurrent at the electrode. Also disclosed is a support comprising an array of nucleic acid probe elements.

### CROSS-REFERENCE TO RELATED APPLICATIONS

This application claims the benefit of the filing date of U.S. Provisional Application No. 60/427,085, filed Nov. 15, 2002, the disclosure of which is incorporated by reference.

### TECHNICAL FIELD

The present application is generally related to the detection of molecules, and more particularly, to the detection of a target nucleic acid sequence.

### SUMMARY OF THE INVENTION

Disclosed herein is a method for detecting a target nucleic acid sequence as well as an apparatus and a kit therefor. The method uses a non-covalent photoelectrochemical compound that preferentially labels double-stranded nucleic acids over single-stranded nucleic acids. The photoelectrochemical intercalator need not be covalently attached or otherwise conjugated to the target nucleic acid sequence.

One aspect provides an apparatus for detecting a target nucleic acid sequence. An embodiment of the system comprises: a support comprising an electrode and a nucleic acid probe attached thereto, wherein the nucleic acid probe comprises a sequence complementary to the target nucleic acid sequence; a non-covalent photoelectrochemical label for contacting with the nucleic acid probe; a light source for irradiating the nucleic acid probe; and a data collection controller for measuring a current at the electrode. Some embodiments of the system optionally further comprise a fluid handling system, a temperature control system, and/or an optical scanner.

Another aspect of the present invention provides a method for detecting a target nucleic acid sequence. An embodiment of the method comprises at least the steps of: contacting a nucleic acid probe with a target nucleic acid and a non-covalent photoelectrochemical label to form a reaction mixture; irradiating the mixture; and observing a photocurrent at the electrode, wherein the photocurrent indicates the presence and/or amount of the target nucleic acid. The nucleic acid probe is attached to an electrode, the nucleic acid probe comprises a sequence complementary to the target nucleic acid sequence, and a support comprises the nucleic acid probe and the electrode

In some embodiments, the method further comprises contacting the nucleic acid probe with a sacrificial reductant or a sacrificial oxidant. In some embodiments, the sacrificial reductant comprises at least one of a tertiary amine, tripropylamine, ethylenediaminetetraacetic acid, and salts thereof.

In some embodiments, the method further comprises maintaining the nucleic acid probe under conditions conducive for nucleic acid hybridization. In some embodiments, the method further comprises washing the nucleic acid probe to remove excess nucleic acid target. In some embodiments, the method further comprises washing the nucleic acid probe to remove excess non-covalent photoelectrochemical label.

Another aspect provides a kit for detecting a target nucleic acid sequence. An embodiment of the kit comprises: a support, wherein the support comprises an electrode and a nucleic acid probe attached thereto, and wherein the nucleic acid probe comprises a sequence complementary to the target nucleic acid sequence; and a non-covalent photoelectrochemical label.

In some embodiments, the nucleic acid probe comprises DNA. In some embodiments, the nucleic acid probe comprises RNA. In some embodiments, the target nucleic acid comprises DNA. In some embodiments, the target nucleic acid comprises RNA.

In some embodiments, the support comprises an array of nucleic acid probe elements. In some embodiments, the array comprises greater than about 10 nucleic acid probe elements.

In some embodiments, the electrode comprising at least one of gold, platinum, silicon, glassy carbon, graphite, indium-tin oxide, and diamond.

In some embodiments, the non-covalent photoelectrochemical label is a compound comprising: a metal comprising at least one of ruthenium, osmium, cobalt, rhodium, nickel, and platinum; and a ligand comprising at least one of polypyridyl ligands, bipy, phen, DIP, dppz, phi, terpy, and derivatives thereof. In some embodiments, the non-covalent photoelectrochemical label comprises a cation selected from the group consisting of [Ru(bipy)₃]²⁺, [Ru(bipy)₂dppz]²⁺, [Ru(phen)₃]²⁺, and combinations thereof.

In some embodiments, the light source is a laser. In some embodiments, the light source is a source of visible light.

Some embodiments further comprise a sacrificial reductant or sacrificial oxidant for contacting with the nucleic acid probe. In some embodiments, the sacrificial reductant is selected from the group consisting of a tertiary amine, tripropylamine, ethylenediaminetetraacetic acid, and salts thereof.

### BRIEF DESCRIPTION OF THE FIGURES

FIG. 1 is a schematic of an embodiment of an apparatus suitable for practicing the photoelectrochemical method disclosed herein.

FIG. 2 illustrates a possible mechanism through which the disclosed method may operate.

FIG. 3 is a flowchart of some embodiments of the disclosed method.

FIG. 4 is the photocurrent measured at an electrode modified with single-stranded DNA and double-stranded DNA in the presence of [Ru(bipy)₃]²⁺ and EDTA.

FIG. 5 is a graph illustrating a relationship between the potential of an electrode modified with double-stranded DNA and the observed photocurrent in the presence of [Ru(bipy)₃]²⁺ and EDTA.

### DETAILED DESCRIPTION OF THE PREFERRED EMBODIMENTS

As used herein, “bipy” is 2,2′-bipyridine, “phen” is 1,10-phenanthroline, “DIP” is 4,7-diphenyl-1,10-phenanthroline, “dppz” is dipyrido[3,2-a:2′,3′-c]phenazine, and “terpy” is 2,2′:6′,2″-terpyridine. These ligands are members of a family of ligands that comprise a plurality of pyridine units and which is referred to herein as “polypyridyl ligands.” The term “phi” represents 9,10-phenanthrenequinone diimine. “Nucleic acid” includes DNA, RNA, PNA, locked nucleic acids (LNA), and combinations thereof, any of which are single and/or double stranded. Double-stranded nucleic acids comprehend homoduplexes, for example DNA-DNA, and heteroduplexes, for example DNA-RNA. The “ss” prefix means “single-stranded,” for example, “ss-DNA” means “single-stranded DNA.” Similarly, “ds” means “double stranded” or duplex. References in this disclosure to DNA are also directed to other nucleic acids, for example, RNA, PNA, LNA, and combinations thereof unless otherwise provided. The term “photoelectrochemical” is also sometimes referred to as “electrochemiluminescent” or “ECL” by those skilled in the art.

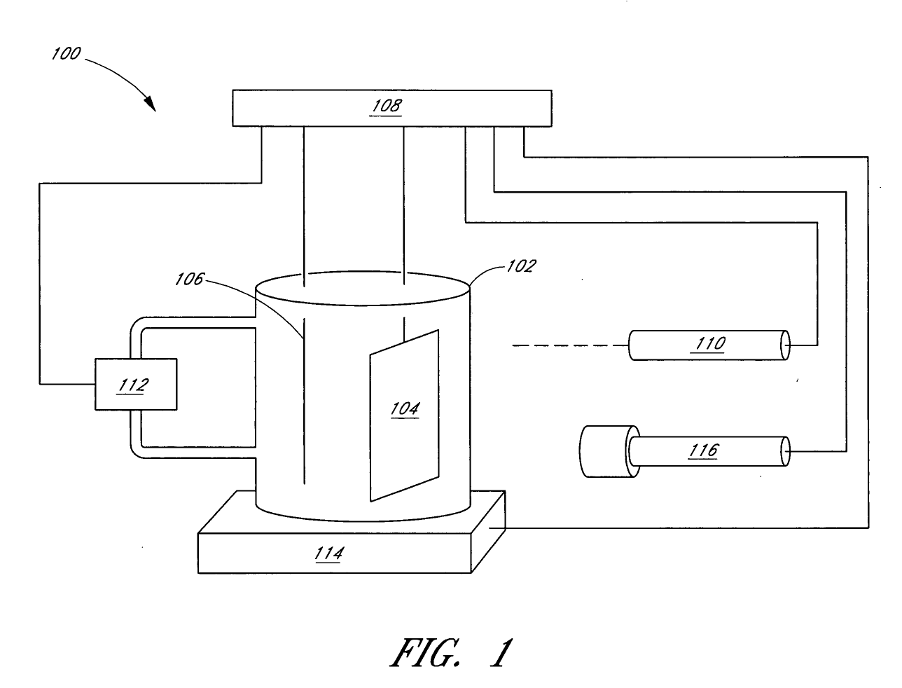

FIG. 1 illustrates an embodiment of an apparatus 100 suitable for practicing the disclosed method. The apparatus 100 comprises an electrochemical cell 102, a working electrode 104, a counter electrode 106, a data capture controller 108, a light source 110, an optional fluid handling system 112, and an optional temperature control system 114. The working electrode 104 is also referred to herein as “the support.”

The electrochemical cell 102 comprises the support 104, the counter electrode 106, and optionally, a reference electrode (not illustrated). The optional reference electrode is of any type known in the art. Some embodiments use a solid reference electrode. In some embodiments, the support 104 comprises one or more of the walls of the electrochemical cell 102. The electrochemical cell 102 optionally comprises fluid handling system 112 of any type known in the art, used, for example, for injecting samples, washing the cell, adding reagents, and the like. In some embodiments, the electrochemical cell 102 is a flow cell. In some embodiments, the electrochemical cell 102 is equipped with a temperature control system 114 of any type known in the art, which is used to heat and/or cool the support 104 and/or electrochemical cell 102. Some embodiments further comprise an optical scanner 116 of any type known in the art, the use of which is discussed in greater detail below.

The support 104 comprises an electrode to which one or more types of nucleic acid probe are attached. The support 104 is described in greater detail below. The counter electrode 106 and optional reference electrode are of any type known in the art that are compatible with the disclosed apparatus and method.

The light source 110 is configured to irradiate the support 104 with electromagnetic radiation. The light source 110 is any source of sufficient energy and intensity to initiate the disclosed photoelectrochemical reaction, which is described in greater detail below. Examples of suitable light sources include an incandescent lamp, a halogen lamp, a light emitting diode (LED), a fluorescent lamp, an arc lamp, the sun, and a laser. In some embodiments, the electromagnetic radiation is infrared radiation, visible light, and/or ultraviolet radiation. In some embodiments, the light source 110 includes an optical system configured to direct electromagnetic radiation to impinge on the support 104 or a portion thereof. In some embodiments, the light source 110 is configured to scan the surface of support 104. In some embodiments, the light source 110 scans the surface of the support 104 through moving the support 104.

The data capture controller 108 is used to control the electrochemical cell 102. In some embodiments, the data capture controller 108 controls the operation of other components of the apparatus 100, as described below. With respect to the electrochemical cell 102, the data capture controller at least comprises the functions of a potentiostat, that is, is able to control and measure the voltage and/or current in the cell 102. Currents on the order of nanoamperes and picoamperes are routinely measured using devices known in the art. Currents as small as single electrons are also measurable, for example, using quantum dots, scanning probe devices, and single electron tunneling (SET) devices. In embodiments in which the data capture controller 108 regulates the light source 110, the data capture controller 108 at least controls the irradiation of the support 104. In some embodiments, the data capture controller 108 also controls which portion of the support 104 is illuminated. For example, in some embodiments, the data capture controller 108 causes the light source 110 to scan the support 104 in a predetermined pattern. This embodiment is useful, for example, in embodiments in which the support comprises an array, as discussed in greater detail below. In some embodiments, the data capture controller 108 optionally receives data from an optical scanner or detector 116 configured to read optical information from the support 104. For example, in some embodiments, the support 104 comprises optically encoded identifying indicia, e.g., a barcode or other indicia discussed below. In other embodiments, discussed below, the optical scanner 116 is configured to detect fluorescence from the support 104. In other embodiments, the data capture controller 108 is configured to read identifying indicia that are coded in another way, for example, using a radio frequency (RF) tag, or using a integrated device, for example, a microprocessor. In some embodiments, the indicia identify a program for the data capture controller. The data capture controller 108 comprises one or more interfaces for data acquisition and/or data input known in the art, for example, a display, keyboard, printer, disk drive, data port, I/O port and the like.

In some embodiments, the data capture controller 108 also controls and acquires data from optional components, including a fluid handling system 112, temperature control system 114, and/or optical scanner 116. The data capture controller 108 is illustrated as a single unit, but may comprise separate units to implement each of, or a combination of, functions. For example, in some embodiments, the data capture controller 108 comprises modular units, thereby permitting a user to add control and data acquisition functions to augment the capabilities of the disclosed apparatus. In some embodiments, the data capture controller 108 is a microprocessor-based device. In some embodiments, the data capture controller 108 is user programmable. In some embodiments, the data capture controller 108 is preprogrammed.

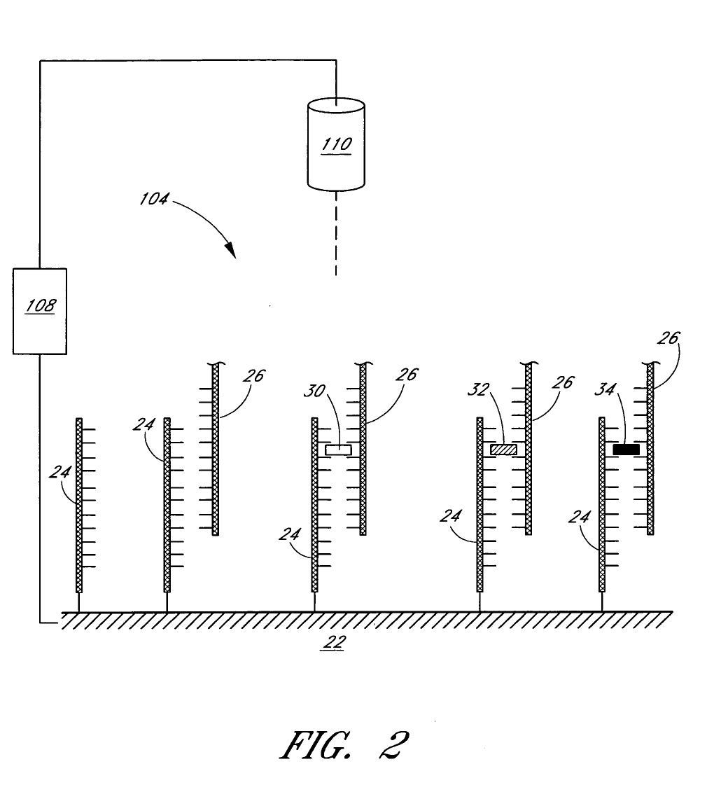

A detailed view of an embodiment of a support 104 is illustrated in FIG. 2. The support 104 comprises an electrode 22 to which a nucleic acid probe 24 is attached. A complementary nucleic acid sequence (“nucleic acid target”) 26 binds to the probe 24. Non-complementary nucleic acid sequences (not shown) do not bind to the probe 24.

The disclosed system uses a non-covalent photoelectrochemical compound that preferentially labels a double-stranded nucleic acid over a single-stranded nucleic acid. Some embodiments use a plurality of the disclosed photoelectrochemical compounds. In some embodiments, the photoelectrochemical compound is a non-covalent label for double-stranded nucleic acid. In the illustrated embodiment, the photoelectrochemical label is a photoelectrochemical intercalator 30 that binds to double-stranded nucleic acids, but does not appreciably bind to single-stranded nucleic acids. Without being bound by any theory, it is believed that irradiating the bound intercalator 30 generates an intercalator in an excited state 32. The excited intercalator 32 is easier to oxidize than the ground-state intercalator 30. Biasing the electrode 22 to a potential sufficient to oxidize the excited intercalator 32, but not the ground-state intercalator 30 causes the oxidation of an excited intercalator 32 to an oxidized intercalator 34. In the illustrated embodiment, the oxidized intercalator 34 remains bound to the double-stranded nucleic acid. In some embodiments, a sacrificial reductant is provided, which reduces the oxidized intercalator 34 back to the ground-state intercalator 30, thereby providing a photocurrent until the sacrificial reductant is exhausted or the irradiation is discontinued.

The electrode 22 is any electrode material known in the art that is compatible with the disclosed assay, for example, gold, platinum, silicon, glassy carbon, graphite, indium-tin oxide, and conducting or semi-conducting diamond. Diamond and indium-tin oxide electrodes are transparent, providing flexibility in the optical arrangement of the components, for example, in embodiments in which the support 104 forms a wall in the electrochemical cell 102. In some embodiments, the electrode 22 is a layer deposited on a substrate using any means known in the art, for example, by physical vapor deposition, chemical vapor deposition, spin coating, printing, etc. The substrate is any suitable substrate, for example, a semiconductor substrate, silicon, glass, silica, or sapphire. The electrode 22 has any suitable shape, for example, rectangular, circular, hexagonal, or any other suitable shape. In the embodiment illustrated in FIG. 1, the electrode 22 is planar. Those skilled in the art will understand that the electrode 22 may be non-planar, for example, cylindrical, tubular, a prism, pyramid, etc. In some embodiments, the entire inner wall of the electrochemical cell 102 is the electrode 22.

The nucleic acid probe 34 is attached to the electrode 22 using methods known in the art. In some embodiments, the nucleic acid probe 34 is attached using an attachment group. The attachment group will depend on the composition of the particular electrode material to be derivatized. For example, derivatizing a gold electrode with a monolayer of nucleic acids terminating in thiol attachment groups is well known in the art, for example, as described in U.S. Pat. No. 5,472,881, the disclosure of which is incorporated by reference. Diamond electrodes have been derivatized with diazonium compounds, as described in Kuo et al. “Electrochemical Modification of Boron-Doped Chemical Vapor Deposited Diamond Surfaces with Covalent Bonded Monolayers” Electrochem. Solid-State Lett. 1999, 2:6, 288-290, the disclosure of which is incorporated by reference. Silicon electrodes have been derivatized using substituted alkoxysilanes, for example, as described in Yi Cui et al, Science 2001, 293, 1289-92, the disclosures of which are incorporated by reference. Glassy carbon electrodes have been derivatized through amines, as described in R. Deinhammer et al. Langmuir 1994, 10, 1306, or carbodiimide, as described in Mikkelsen et al., Electroanalysis 1992, 4, 929, the disclosures of which is incorporated by reference. Electrodes have also been derivatized through carboxylate-amine functional groups and biotin-avidin coupling.

In some embodiments, the support 104 comprises an array or microarray of nucleic acid probe elements, which is useful in detecting a plurality of target nucleic acid sequences. Such arrays and microarrays are sometimes also referred to as “chips.” Each array element comprises a nucleic acid probe 24. The probes may comprise elements of any type of nucleotide or analog thereof, such as DNA, RNA, PNA, and/or locked nucleic acids (LNA), or any combination thereof. Because the nucleic acid probe elements of the array are addressable optically, an array may be constructed in which each array element need not be addressable electrically. The consequence of this optical addressability is that an entire nucleic acid array may be laid-down on a single electrode, thereby simplifying construction. In some embodiments, the array is patterned on a plurality of electrodes. In some embodiments, the array is patterned on one surface of the support 104. In some embodiments, the array is patterned on a plurality of surfaces of the support 104, for example, on both sides of a planar support 104.

In constructing the array, the nucleic acid probes 24 are applied to the electrode 22 by any means known in the art, for example, by pipette, ink-jet printing, photolithography, or contact printing. In assembling the array, the probes 24 are attached to the electrode as described above. As used herein, an array element or element is an addressable unit of the array. An element may comprise one or more probes. An array comprises at least two elements. Arrays comprising up to about 10⁹ elements have been reported. Accordingly, the number of elements in an array constructed according to the present disclosure may be about 10, 100, 1000, 10,000, 100,000, 10⁶, 10⁷, 10⁸, 10⁹, or more. The spacing of the array elements may be uniform or may vary. For arrays with a large number of elements, a closer interelement spacing permits the construction of a physically smaller array. Center-to-center element spacings on the order of 100 μm are common. Spacings of about 10 μm, 1 μm, and smaller have been reported. The present disclosure contemplates arrays with interelement spacings any or all of these values. By way of example, arrays of over 10,000 elements are routinely fabricated on standard microscope slides. A 100-μm center-to-center spacing of the array elements provides an array of over 100,000 elements in a standard 96-well microtiter plate.

In some embodiments, the array elements form a square grid, a rectangular grid, a hexagonal grid, or any pattern or combination of patterns desired. In some embodiments, the array elements are laid-down randomly, then interrogated to determine their spatial addresses. Any spatial arrangement of array elements may be used, so long as the elements are addressable. The size of the elements depends on the particular application. For example, detection of small amounts of nucleic acid may require small array elements. Construction of microarrays will similarly use small elements. A single array may comprise elements of different sizes. Contact printing can deposit a sample of a solution on the order of nanoliters. One of ordinary skill will appreciate that the size of an element is limited by the interelement spacing.

The surface of the array may be substantially smooth or may have features. For example, the array elements may be applied in depressions or on raised features. Furthermore, a plurality of array elements may be placed on each depressed or raised feature. In the example cited above in which an array with 10,000 elements was formed on a standard 96-well plate, each well contained about 100 elements. These depressed or raised areas may serve to provide a particular environment for the array elements, for example, by retaining a solution or by facilitating drying.

The support may further possess indicia or markings to provide or assist in addressability of the array elements. The depressed or raised features described above may also serve this purpose. The markings or indicia may also serve to orient the support 104 in the apparatus, or to identify it, for example, using a barcode or another machine-readable marking. The indicia are applied to any convenient location, which may be on a surface of the support 104 on which the array is applied or on a different surface. The location is selected to be readable by the optical scanner 116. In some embodiments, the support 104 comprises another machine-readable identification, for example, an RF-tag or integrated device (e.g., a microprocessor).

A laser is a useful light source 110 for a system that comprises small array elements, although any sufficiently focused light source may be used. Methods for scanning an array using a laser are well known in the art.

In some embodiments, the support 104 is provided with a means for determining the position that the light source is irradiating in conjunction with the optical scanner 116. For example, the electrode itself may have a grid or other reference marks or indices. Methods for producing such reference marks will vary with the type of electrode and are known to those skilled in the art, for example, lithography, electrochemical etching, ion-assisted etching, electroplating, physical vapor deposition, and chemical vapor deposition. Alternatively, such indicia may be part of the array pattern. For example, a colored, fluorescent, or photoelectrochemical molecule could be attached to the electrode as an array element. In some embodiments, the index is a nucleic acid probe. An index target known to bind to the index probe is added and hybridized to the probe. As the light source scans the array, it will generate a photocurrent when each of these index elements is irradiated. In some embodiments, the index target is conjugated to a colored, fluorescent, or photoelectrochemical molecule.

In some embodiments, the support 104 comprises an element that generates an electrical signal when irradiated, for example, a photovoltaic, photoresistive, or another light-sensitive element known in the art.

In embodiments in which the photoelectrochemical intercalator is fluorescent, array elements that comprise double-stranded nucleic acids to which the intercalator is bound may be determined optically, thereby increasing the system throughput by scanning only fluorescent array elements.

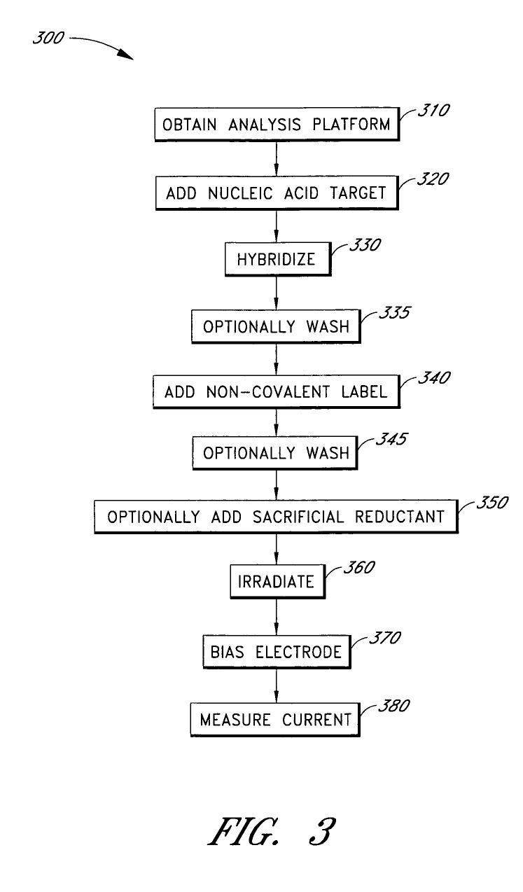

FIG. 3 provides a method 300 for detecting a nucleic acid sequence with reference to the support illustrated in FIG. 2. In step 310, a support 104 is obtained. In step 320, the nucleic acid probe 24, which is attached to the support 104, is contacted with a nucleic acid target 26. In step 330, the nucleic acid probe 24 is subjected to conditions conducive to hybridization. If the probe 24 and target 26 have complementary sequences, they will hybridize, thereby forming a double-stranded nucleic acid. Otherwise, they will not hybridize appreciably. The support 104 is then optionally washed to remove unhybridized target 26 in step 335. In step 340, the nucleic acid probe 24 is contacted with a photoelectrochemical intercalator 30. The intercalator 30 binds to a double-stranded nucleic acid formed in the hybridization step. If the probe 24 remains single stranded, i.e. does not hybridize, the intercalator 30 does not appreciably bind. In step 345, the support 104 is optionally washed to remove unbound intercalator 30. In step 350, the nucleic acid probe 24 is optionally contacted with a sacrificial reductant. The nucleic acid probe 24, nucleic acid target 26, photoelectrochemical intercalator 30, and optional sacrificial reductant together form a reaction mixture, which may further comprise additional components. In step 360, the reaction mixture is irradiated with electromagnetic radiation with an energy and intensity sufficient to excite bound intercalator in the ground-state 30 into an excited state intercalator 32 that is easier to oxidized than the ground-state intercalator. In step 370, the electrode 22 is biased to a potential that will oxidize an intercalator in the excited state 32, but not an intercalator in the ground-state 30, to an oxidized intercalator 36. The oxidation of the excited state intercalator 32 generates a photocurrent. In step 380, the photocurrent is measured.

In step 310, a support 104 is obtained. The disclosed method is practiced using any nucleic acid support 104 disclosed herein. In some embodiments, the support 104 comprises an array or microarray of nucleic acid probes.

In step 320, the nucleic acid target 26 is contacted with the electrode-bound nucleic acid probe 24 on the support 104. Typically, the nucleic acid target 26 is dissolved in a solvent, for example, water, an aqueous buffer, an organic solvent, or another suitable solvent. In some embodiments, the nucleic acid target 26 is contacted with the nucleic acid probe 24 using a fluid handling system 112. In some embodiments, the fluid handling system 112 is controlled by the data capture controller 108.

In step 330, the target 26 and probe 24 are allowed to hybridize. In some embodiments, the hybridization conditions are established and maintained using the data capture controller 108 in concert with a fluid handling system 112 and/or a temperature control system 112 as described above.

Conditions conducive to nucleic acid hybridization are known in the art and will differ depending on factors including the nature of the nucleic acid target 26 and probe 24, the ionic strength of the solvent, and the identity of the solvent. For example, hybridizing a single-stranded DNA target to a single-stranded DNA probe will require different conditions than hybridizing a single-stranded RNA target to a single-stranded DNA probe. Either or both the target and probe may also be double stranded, in which case the double-stranded nucleic acid is denatured prior to the hybridization step. The probe and target need hybridize only along a portion of their respective lengths, i.e., either or both the probe and target may have single-stranded portions after the hybridization step. Optimum hybridization conditions will be typically determined by the melting properties (Tm) of the nucleic acid and the ionic strength of the hybridization solution. The hybridization rate is generally optimal about 20-30° C. below the Tm, although temperatures slightly below, at, or even above Tm may also be useful in certain cases. The wash conditions provided in typical protocols serve to maximize stringency, i.e. to prevent hybridization with nucleic acid that is not completely complementary (if complete complementarity is required). If high stringency is not a requirement, the wash temperature can be reduced and/or the ionic strength of the wash buffer can be raised. “Stringency” of hybridization reactions is readily determinable through routine optimization, and generally is an empirical calculation dependent upon the lengths of the nucleic acid strands, washing temperature, and salt concentration. In general, longer strands require higher temperatures for proper annealing, while shorter strands require lower temperatures. Hybridization generally depends on the ability of denatured nucleic acids to anneal when complementary strands are present in an environment below their melting temperature. The higher the degree of desired homology between the probe and target, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley; 1995, the disclosure of which is incorporated by reference.

In step 340, the nucleic acid probe 24 is contacted with a photoelectrochemical intercalator 30. In some embodiments, the photoelectrochemical intercalator 30 is contacted with the nucleic acid probe 24 using a fluid handler 112, which is optionally controlled using the data capture controller 108. Some embodiments use a plurality of photoelectrochemical intercalators 30. In some embodiments, the nucleic acid probe 24 is simultaneously contacted with the target nucleic acid (step 320) and with the photoelectrochemical intercalator 30 (step 340).

The photoelectrochemical intercalator 30 is selected to bind to duplex nucleic acid formed in the hybridization step, but not to the single-stranded probe 24 or target 26. The other requirement is that the intercalator 30 is photoelectrochemically active. Any intercalator that meets these criteria may be used. The intercalator may be organic, inorganic, or organometallic. Organic intercalators include ethidium bromide and related compounds. Metal complexes of ruthenium, osmium, cobalt, rhodium, nickel, and platinum in which at least one ligand is bipy, phen, DIP, dppz, phi, terpy, or derivatives thereof are suitable photoelectrochemical intercalators. Examples of such complexes are disclosed in U.S. Pat. Nos. 5,112,974, 4,699,987, 4,980,473, and Erkkila et al. “Recognition and Reaction of Metallointercalators with DNA” Chem. Rev., 1999, 99, 2777, the disclosures of which are incorporated by reference. U.S. Pat. No. 4,980,473, the disclosure of which is incorporated by reference, discloses that enantiomers of [Ru(DIP)₃]²⁺ differentially bind Z- and B-DNA.

The tris(polypyridyl) ruthenium compounds—particularly those in which the cation is [Ru(bipy)₃]²⁺, [Ru(bipy)₂dppz]²⁺, or [Ru(phen)₃]²⁺—are useful as photoelectrochemical intercalators. The osmium analogs are also useful photoelectrochemical intercalators.

When the photoelectrochemical intercalator 30 is added to a hybridized probe-target, it binds to the double-stranded nucleic acid, but does not bind to a single-stranded nucleic acid. Accordingly, the intercalator 30 will not bind significantly to any probe nucleic acid 24 that is not hybridized to target nucleic acid 26. As such, the intercalator 30 is a universal label for double-stranded nucleic acids, with the added advantage that it need not be covalently attached. A double-stranded DNA-intercalator species is referred to as “intercalator-labeled DNA” herein.

Ruthenium polypyridyl intercalators typically bind to double-stranded nucleic acids randomly. The concentration of the photoelectrochemical intercalator necessary to optimize the photoelectrochemical signal depends on factors including the overlap length between the probe 24 and target 26, the amount of probe 24 and target 26, the efficiency of the photoexcitation of the intercalator 30 to the excited state 32, the efficiency of the oxidation of the excited intercalator 32 to the oxidized form 34, and the concentration of a sacrificial reductant, if present. This concentration is readily determined by one of ordinary skill. The assay may be calibrated to provide quantitative results. Moreover, one may select an intercalator that will selectively bind to double-stranded nucleic acids with specific structural features. As noted above, the enantiomers of certain intercalators are known to select for either B- or Z-DNA. It is also possible to select for base-pair mismatches using the intercalators described in U.S. Pat. No. 6,031,098, the disclosure of which is incorporated by reference.

In step 350, the nucleic acid probe 24 is optionally contacted with a sacrificial reductant. The sacrificial reductant is capable of reducing an oxidized intercalator 34 to the ground state intercalator 30, thereby permitting the intercalator 30 to generate a photocurrent as long as it is irradiated, which facilitates the detection and measurement of the photocurrent. A suitable sacrificial reductant does not interfere with the assay to an extent that the assay is unusable. In some embodiments, the electrode 22 does not directly oxidize the sacrificial reductant, thereby reducing potentially interfering background currents. The sacrificial reductant will depend on the particular photoelectrochemical intercalator used. Suitable sacrificial reductants for ruthenium-based intercalators include tertiary amines in general, and tripropylamine (TPA) and ethylenediaminetetraacetic acid (EDTA) in particular. Another suitable sacrificial reductant is a β-lactam.

The nucleic acid probe 24, nucleic acid target 26, photoelectrochemical intercalator 30, and optional sacrificial reductant together form a reaction mixture, which may further comprise additional components.

Other embodiments, described in greater detail below, use a sacrificial oxidant instead of a sacrificial reductant. Other embodiments use neither a sacrificial reductant nor sacrificial oxidant.

In step 360, the reaction mixture is irradiated using a light source 110, which is optionally regulated using the data capture controller 108. The energy and intensity of light is selected to excite an intercalator 30 bound to the double-stranded region of a hybridized probe-target into an excited state intercalator 32. In some embodiments, the apparatus is configured to permit scanning the surface of the support 104 with the light source 110. In embodiments in which the support 104 comprises an array of nucleic acid probes; the apparatus is configured to irradiate each array element individually. Because the base sequences of the nucleic acid probes 24 that make up the array elements are known, one may identify a portion of the sequence of any target 26 that binds to a probe by the position of the probe in the array.

In step 370, the electrode 22 is biased to an oxidizing potential with respect to the excited intercalator 32. The potential of the electrode 22 is controlled by the data capture controller 108. In some embodiments, the electrode 22 is biased to a potential capable of oxidizing the excited intercalator 32, but not an intercalator in the ground state 30. In some embodiments, steps 360 and 370 overlap in time.

In some embodiments, the electrode 22 is biased to a potential capable of oxidizing the ground state intercalator 30. This embodiment is useful in cases in which optical addressing is not necessary, for example, where the support 104 comprises only one type of nucleic acid probe 24. This assay is an electrochemical assay rather than a photoelectrochemical assay.

In some embodiments, discussed in greater detail below, the electrode 22 is biased to a reducing potential.

In step 380, the current at the electrode 22 is measured using any means known in the art. In the illustrated embodiment, the data capture controller 108 is configured to measure the current. In embodiments using a sacrificial reductant, the photocurrent continues until the sacrificial reductant is consumed or the irradiation is terrninated. In embodiments not using a sacrificial reductant, the photocurrent continues until all of the excited intercalator 32 is oxidized.

In some embodiments, the electrode is reusable for additional assays. In some embodiments, the support 104 is simply subjected to denaturing conditions and the denatured single-stranded nucleic acids washed from the electrode.

Without being bound by any theory, the following describes the mechanism believed to be operative in a system using [Ru(bipy)₃]²⁺ as the photoelectrochemical intercalator. Similar mechanisms are believed to apply to other photoelectrochemical intercalators as well. In the following description, the ground state intercalator 30 is [Ru(bipy)₃]²⁺, the excited-state intercalator 32 is [Ru(bipy)₃]²⁺*, and the oxidized intercalator 34 is [Ru(bipy)₃]³⁺. Irradiating [Ru(bipy)₃]²⁺ generates the excited species, [Ru(bipy)₃]²⁺*, which is more easily oxidized to [Ru(bipy)₃]³⁺ than [Ru(bipy)₃]²⁺. The precise nature of the excited species is not critical to the method, and the photoelectron may be metal-centered or ligand-centered. [Ru(bipy)₃]²⁺ intercalates double-stranded regions of a target 26 hybridized to a probe nucleic acid 24 attached to an electrode 22. Irradiating the intercalator excites the intercalator to an excited state, [Ru(bipy)₃]²⁺*.

In some embodiments, the electrode 22 is biased to a potential that oxidizes [Ru(bipy)₃]²⁺*, but not [Ru(bipy)₃]²⁺, to [Ru(bipy)₃]³⁺. Irradiating the intercalator-labeled nucleic acid generates intercalated [Ru(bipy)₃]²⁺*, which is oxidized to by the electrode 22 to [Ru(bipy)₃]³⁺, generating a photocurrent. If the probe 24 is not hybridized, no intercalator 30 will bind and no photocurrent will be observed.

Without being bound by any theory, it is believed that the photoelectron quantum mechanically tunnels from the excited [Ru(bipy)₃]²⁺* to the electrode 22. Because the rate of tunneling depends on the distance between the [Ru(bipy)₃]²⁺* and the electrode 22, a photocurrent is generated only by [Ru(bipy)₃]²⁺* molecules that are close to the electrode surface. Typically, only those [Ru(bipy)₃]²⁺* molecules bound to target-probe duplexes attached to the surface of the electrode 22 are close enough to the electrode to generate a photocurrent. Other studies indicate that the DNA double helix itself facilitates electron transfer. For example, see Murphy et al. “Fast Photoinduced Electron Transfer Through DNA Intercalation” Proc. Natl. Acad. Sci, USA, 1994, 91, 5315, the disclosure of which is incorporated by reference.

In other embodiments, a sacrificial reductant is added that reduces [Ru(bipy)₃]³⁺ to [Ru(bipy)₃]²⁺, but does not reduce [Ru(bipy)₃]²⁺ to [Ru(bipy)₃]¹⁺. In this embodiment, the electrode 22 is biased to a potential that oxidizes [Ru(bipy)₃]²⁺* but not [Ru(bipy)₃]²⁺ to [Ru(bipy)₃]³⁺. Irradiating the intercalator-bound nucleic acid generates intercalated [Ru(bipy)₃]²⁺*, which is oxidized to by the electrode to [Ru(bipy)₃]³⁺, generating a photocurrent. The sacrificial reductant reduces intercalated [Ru(bipy)₃]³⁺ to back to [Ru(bipy)₃]²⁺. Because [Ru(bipy)₃]²⁺ is regenerated in this embodiment, the photocurrent continues until the sacrificial reductant is consumed or the irradiation is terminated. Again, if the probe is not hybridized, no intercalator 30 will bind and no photocurrent will be observed.

In still other embodiments, a sacrificial oxidant that oxidizes [Ru(bipy)₃]²⁺* but not [Ru(bipy)₃]²⁺ to [Ru(bipy)₃]³⁺ is added to the system. The electrode is biased to a potential that reduces [Ru(bipy)₃]³⁺ to [Ru(bipy)₃]²⁺, but not to [Ru(bipy)₃]¹⁺. Irradiating an intercalator-bound nucleic acid generates intercalated [Ru(bipy)₃]²⁺*, which is oxidized to by the sacrificial oxidant to [Ru(bipy)₃]³⁺. The electrode reduces intercalated [Ru(bipy)₃]³⁺ to back to [Ru(bipy)₃]²⁺, generating a photocurrent. Because [Ru(bipy)₃]²⁺ is regenerated in this embodiment, the photocurrent continues until the sacrificial oxidant is consumed or the irradiation is terminated. Again, if the probe is not hybridized, no intercalator 30 will bind and no photocurrent will be observed. Any sacrificial oxidant that does not otherwise interfere with the assay may be used. [Co(C₂O₄)]³⁻ and hydrazine are suitable sacrificial oxidants.

The foregoing discusses photoelectrochemical intercalators that undergo photo-assisted oxidation. Those skilled in the art will realize that photoelectrochemical intercalators that undergo photo-assisted reduction may also be used within the scope of the disclosed invention.

### EXAMPLE

A three-electrode electrochemical cell with a Au working electrode, a Pt wire counter electrode, and a Ag/AgCl reference electrode was connected to a potentiostat set up to control the potential bias and measure the current. The light source was a 405-nm diode laser, focused to a 50-μm beam size through a pinhole and a set of optical lenses on the working electrode surface. A computer was used to modulate the laser by a square wave (off-on), and to acquire the data through a data acquisition board.

The gold working electrode was cleaned with a 70/30-v/v solution of concentrated sulfuric acid and 30% hydrogen peroxide, then thoroughly rinsed with deionized water. The thiolated probe oligonucleotide X20 (thiol-AACCAGGATTATCCGCTCAC) was deposited on the surface of the electrode, and the electrode thoroughly rinsed with deionized water. This probe self-assembles into a monolayer on gold surfaces. A solution of the target oligonucleotide T20 (GTGAGCGGATAATCCTGGTT) in 1×SSC buffer was added to the electrode and allowed to hybridize at 38° C. The electrode was rinsed with 1×SSC buffer to remove any excess target. A 1-mM solution of [Ru(bipy)₃]Cl₂ in 1×SSC buffer was added to the electrode and the electrode rinsed with 1×SSC buffer. Photoelectrochemical detection was performed in 50 mM EDTA and 0.1 mM NaCl. The electrode potential was 0-0.7 V vs. the Ag/AgCl reference electrode.

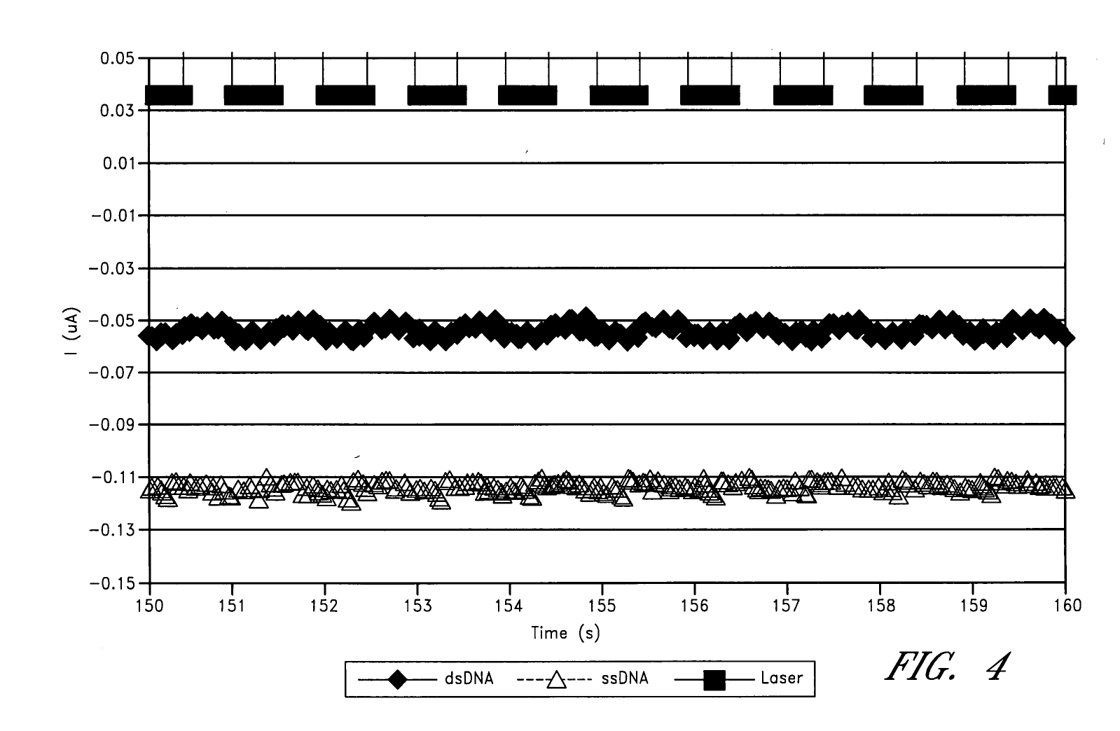

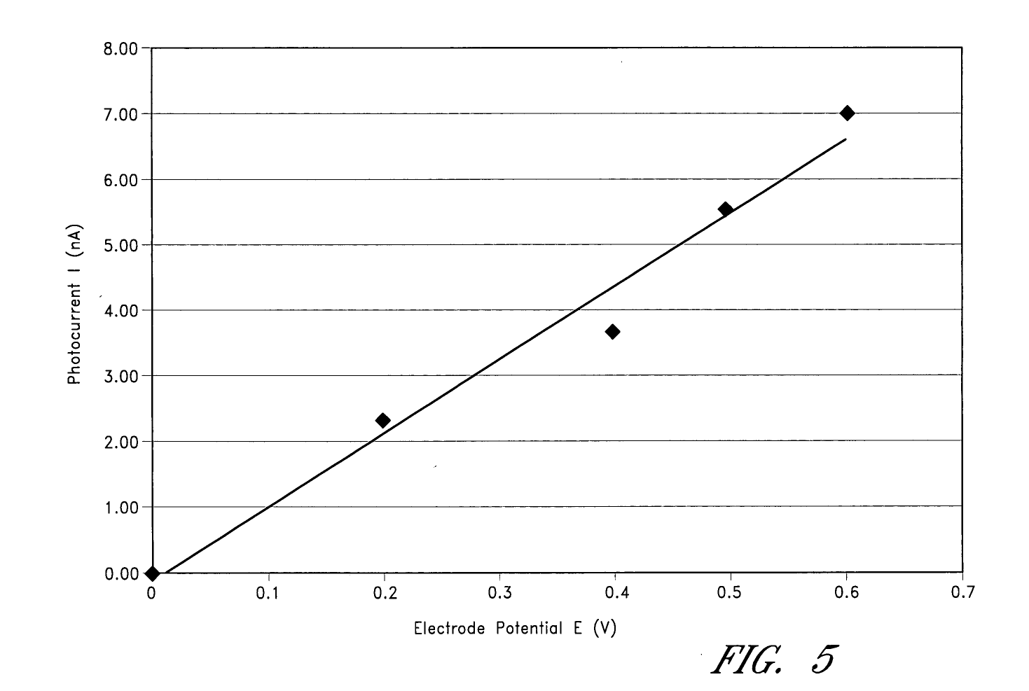

A run in which the laser was modulated at 1 Hz, the data collected at 32 Hz, and the electrode potential was 0.5 V generated the data provided in FIG. 4. In the single-stranded DNA data, the current does not change appreciably with modulation of the laser. The current is coupled with the modulation of the laser in the double-stranded DNA data. FIG. 5 illustrates the dependence of the photocurrent on the potential at the electrode.

The embodiments illustrated and described above are provided as examples only. Various changes and modifications can be made to the embodiments presented herein by those skilled in the art without departure from the spirit and scope of the teachings herein.

What is claimed is:

1. A system for detecting a target nucleic acid sequence comprising:
a support comprising an electrode and a nucleic acid probe attached thereto, wherein the nucleic acid probe comprises a sequence complementary to the target nucleic acid sequence;
a non-covalent photoelectrochemical label for contacting with the nucleic acid probe;
a light source for irradiating the nucleic acid probe; and
a data collection controller for measuring a current at the electrode.

2. The system of claim 1, wherein the nucleic acid probe comprises DNA.

3. The system of claim 1, wherein the nucleic acid probe comprises RNA.

4. The system of claim 1, wherein the target nucleic acid sequence comprises a DNA sequence.

5. The system of claim 1, wherein the target nucleic acid sequence comprises an RNA sequence.

6. The system of claim 1, wherein the support comprises an array of nucleic acid probe elements.

7. The system of claim 6, wherein the array comprises greater than about 10 nucleic acid probe elements.

8. The system of claim 1, wherein the electrode comprises at least one of gold, platinum, silicon, glassy carbon, graphite, indium-tin oxide, and diamond.

9. The system of claim 1, wherein the non-covalent photoelectrochemical label is a compound comprising:
a metal comprising at least one of ruthenium, osmium, cobalt, rhodium, nickel, and platinum; and
a ligand comprising at least one of polypyridyl ligands, 2,2′-bipyridine, 1,10-phenanthroline, 4,7-diphenyl-1,10-phenanthroline, dipyrido[3,2-a:2′,3′-c]phenazine, 9,10-phenanthrenequinone diimine,2,2′:6′,2″-terpyridine, and derivatives thereof.

10. The system of claim 9, wherein the non-covalent photoelectrochemical label comprises a cation is selected from the group consisting of [Ru(bipy)₃]²⁺, [Ru(bipy)₂dppz]²⁺, [Ru(phen)₃]²⁺, and combinations thereof.

11. The system of claim 1, wherein the light source is a laser.

12. The system of claim 1, wherein the light source radiates visible light.

13. The system of claim 1, further comprising a sacrificial reductant for contacting with the nucleic acid probe.

14. The system of claim 1, wherein the sacrificial reductant comprises at least one of a tertiary amine, tripropylamine, ethylenediaminetetraacetic acid, and salts thereof.

15. The system of claim 1, further comprising an optical scanner for scanning the support.

16. The system of claim 1, further comprising a fluid handling system for the support.

17. The system of claim 1, further comprising a temperature control system for the support.

18. The system of claim 1, wherein the support further comprises machine readable identifying indicia.

19. A method for detecting a target nucleic acid sequence comprising:
contacting a nucleic acid probe with a target nucleic acid and a non-covalent photoelectrochemical label to form a reaction mixture, wherein
the nucleic acid probe is attached to an electrode,
the nucleic acid probe comprises a sequence complementary to the target nucleic acid sequence, and
a support comprises the nucleic acid probe and the electrode; irradiating the mixture; and
observing a photocurrent at the electrode, wherein the photocurrent indicates the presence and/or amount of the target nucleic acid.

20. The method of claim 18, wherein the nucleic acid probe comprises DNA.

21. The method of claim 18, wherein the nucleic acid probe comprises RNA.

22. The method of claim 18, wherein the target nucleic acid comprises DNA.

23. The method of claim 18, wherein the target nucleic acid comprises RNA.

24. The method of claim 18, wherein the support comprises an array of nucleic acid probe elements.

25. The method of claim 18, wherein the array comprises greater than about 10 nucleic acid probe elements.

26. The method of claim 18, wherein the electrode comprises at least one of gold, platinum, silicon, glassy carbon, graphite, indium-tin oxide, and diamond.

27. The method of claim 18, wherein the non-covalent photoelectrochemical label is a compound comprising:
a metal comprising at least one of ruthenium, osmium, cobalt, rhodium, nickel, and platinum; and
a ligand comprising at least one of polypyridyl ligands, 2,2′-bipyridine, 1,10-phenanthroline, 4,7-diphenyl-1,10-phenanthroline, dipyrido[3,2-a:2′,3′-c]phenazine, 9,10-phenanthrenequinone diimine,2,2′:6′,2″-terpyridine, and derivatives thereof.

28. The method of claim 27, wherein the non-covalent photoelectrochemical label comprises a cation is selected from the group consisting of [Ru(bipy)₃]²⁺, [Ru(bipy)₂dppz]²⁺, [Ru(phen)₃]²⁺, and combinations thereof.

29. The method of claim 18, wherein the nucleic acid probe is irradiated using a laser.

30. The method of claim 18, wherein the nucleic acid probe is irradiated with visible light.

31. The method of claim 18, further comprising contacting the nucleic acid probe with a sacrificial reductant or sacrificial oxidant.

32. The method of claim 30, wherein the sacrificial reductant comprises at least one of a tertiary amine, tripropylamine, ethylenediaminetetraacetic acid, and salts thereof.

33. The method of claim 30, further comprising maintaining the nucleic acid probe under conditions conducive for nucleic acid hybridization.

34. The method of claim 30, further comprising washing the nucleic acid probe to remove excess nucleic acid target.

35. The method of claim 30, further comprising washing the nucleic acid probe to remove excess non-covalent photoelectrochemical label.

36. A kit for detecting a target nucleic acid sequence comprising:
a support comprising an electrode and a nucleic acid probe attached thereto, wherein the nucleic acid probe comprises a sequence complementary to the target nucleic acid sequence; and
a non-covalent photoelectrochemical label.

37. The kit of claim 36, wherein the nucleic acid probe comprises DNA.

38. The kit of claim 36, wherein the nucleic acid probe comprises RNA.

39. The kit of claim 36, wherein the support comprises an array of nucleic acid probe elements.

40. The kit of claim 36, wherein the electrode comprises at least one of gold, platinum, silicon, glassy carbon, graphite, indium-tin oxide, and diamond.

41. The kit of claim 36, wherein the non-covalent photoelectrochemical label is a compound comprising:
a metal comprising at least one of ruthenium, osmium, cobalt, rhodium, nickel, and platinum; and
a ligand comprising at least one of polypyridyl ligands, 2,2′-bipyridine, 1,10-phenanthroline, 4,7-diphenyl-1,10-phenanthroline, dipyrido[3,2-a:2′,3′-c]phenazine, 9,10-phenanthrenequinone diimine, 2,2′:6′,2″-terpyridine, and derivatives thereof.

42. The kit of claim 41, wherein the non-covalent photoelectrochemical label comprises a cation is selected from the group consisting of [Ru(bipy)₃]²⁺, [Ru(bipy)₂dppz]²⁺, [Ru(phen)₃]²⁺, and combinations thereof.

43. The kit of claim 36, further comprising a sacrificial reductant or sacrificial oxidant.

44. The kit of claim 43, wherein the sacrificial reductant comprises at least one of a tertiary amine, tripropylamine, ethylenediaminetetraacetic acid, and salts thereof.

45. The kit of claim 36, wherein the support further comprises machine readable identifying indicia.


SEQUENCE LISTING (2 sequences)

SEQ ID NO: 1 - DNA, 20 bp, Artificial Sequence
  misc_feature 1: n=thiol-a
naccaggattatccgctcac

SEQ ID NO: 2 - DNA, 20 bp, Artificial Sequence
  Synthetic DNA
gtgagcggataatcctggtt